(19) United States
(12) Patent Application Publication
(10) Pub. No.: US 2010/0008821 A1
(43) Pub. Date: Jan. 14, 2010

(54) METHOD AND APPARATUS FOR STERILIZING AND DEODORIZING AIR

(76) Inventor: Patrick Potter, Hamilton (NZ)

Correspondence Address: CHRISTIE, PARKER & HALE, LLP, PO BOX 7068, PASADENA, CA, 91109-7068, US

(21) Appl. No.: 12/503,027

(22) Filed: Jul. 14, 2009

Related U.S. Application Data
(60) Provisional application No. 61/080,466, filed on Jul. 14, 2008.

(51) Int. Cl. F01N 3/10; A61L 9/00; F23J 1/02

(52) U.S. Cl. 422/4; 422/173; 110/266

(57) ABSTRACT

An apparatus for sanitizing and deodorizing air. The apparatus includes: a chamber including an inlet adapted to receive contaminated air, an outlet adapted to transport heated air, a baffle adapted to increase the length of a path traveled by air in the chamber, and an ash removal device adapted to remove ash from the chamber; and at least one combustor, wherein the combustor is adapted to combust a fuel to heat the air in the chamber so that the air is sterilized and deodorized, thereby generating such ash.

### CROSS-REFERENCE TO RELATED APPLICATION(S)

This application claims priority to and the benefit of U.S. Provisional Patent Application No. 61/080,466, filed on Jul. 14, 2008, in the United States Patent and Trademark Office, the entire content of which is incorporated herein by reference. The entire content of United States Patent Applications PROCESS AND APPARATUS FOR DRYING AND POWDERIZING MATERIAL (Attorney Docket No: T643-64003; application Ser. No. ______), HEAT RECOVERY AND PRESSURE CONTROL UNIT (Attorney Docket No: T643-64004; application Ser. No. ______), and ENERGY RECOVERY AND TRANSFER SYSTEM AND PROCESS (Attorney Docket No: T643-63990; application Ser. No. ______) filed on Jul. 14, 2009 in the United States Patent and Trademark Office is incorporated herein by reference.

### FIELD OF THE INVENTION

The present invention relates a method and apparatus for sterilizing and deodorizing air.

### BACKGROUND OF THE INVENTION

Animal byproduct meals, fecal material, agricultural fertilizer, corn byproducts, wheat byproducts, wood pulp, and the like are high moisture content materials that may provide a rich source of energy when effectively dehydrated. Further, some of this material should be sterilized and deodorized before being discharged into the environment. However, animal meal contains a high level of moisture. Further, sewage is transported in water and this water must be removed by pressing the sewage, and the solids that remain after the pressing still contain about 70% to about 80% moisture and about 20% solids by weight. Corn byproducts, wheat byproducts, and wood pulp are other examples of materials that are a good source of energy but generally contain too much moisture to be useable as fuel in their raw state. These materials must be dried to about 5% moisture to be a high grade fuel. A large quantity of high temperature air is required to evaporate the moisture from the material, and the air may become contaminated with odors and pathogens from the material.

Therefore, there is a need for a method and apparatus for sterilizing and deodorizing air.

### SUMMARY OF THE INVENTION

An embodiment of the present invention provides an apparatus for sanitizing and deodorizing air. The apparatus includes: a chamber including an inlet adapted to receive contaminated air, an outlet adapted to transport heated air, a baffle adapted to increase the length of a path traveled by air in the chamber, and an ash removal device adapted to remove ash from the chamber; and at least one combustor, wherein the combustor is adapted to combust a fuel to heat the air in the chamber so that the air is sterilized and deodorized, thereby generating such ash.

The ash removal device may include a blower adapted to blow the ash to an edge of a floor of the chamber. The blower may include at least one air conduit, and at least one nozzle coupled to the at least one air conduit and adapted to blow the ash to the edge. An opening of the at least one nozzle may be oval-shaped so that air from the nozzle is directed across the floor. The ash removal device may include an auger adapted to transport the ash out of the chamber.

The at least one combustor may include a combustor adapted to combust powdered organic fuel. The at least one combustor may include an alternate combustor adapted to combust an alternate fuel.

Another embodiment of the present invention provides a method for sanitizing and deodorizing air. The method includes: receiving contaminated air into a chamber through an inlet; heating the contaminated air in the chamber by combusting fuel with at least one combustor to sterilize and deodorize the air, thereby generating ash, wherein the chamber includes a baffle adapted to increase the length of a path traveled by the air in the chamber; transporting the heated air out of the chamber through an outlet, and removing the ash from the chamber with an ash removal device.

The removing of the ash may include blowing the ash to an edge of a floor of the chamber with a blower. The blower may include at least one air conduit, and at least one nozzle coupled to the at least one air conduit and adapted to blow the ash to the edge. An opening of the at least one nozzle may be oval-shaped so that air from the nozzle is directed across the floor. The removing of the ash may further include transporting the ash out of the chamber with an auger.

The at least one combustor may include a combustor adapted to combust powdered organic fuel. The at least one combustor may include an alternate combustor adapted to combust an alternate fuel.

An embodiment of the present invention provides an apparatus for removing ash. The apparatus includes: a chamber including an inlet adapted to receive air, a combustor adapted to heat the air, thereby generating such ash, an outlet adapted to transport heated air, and a blower adapted to blow the ash to an edge of a floor of the chamber wherein the blower comprises at least one conduit, and at least one nozzle coupled to the at least one conduit and adapted to blow the ash to the edge.

An opening of the at least one nozzle may be oval-shaped so that a medium from the nozzle is directed across the floor. The chamber may further include an auger adapted to transport the ash out of the chamber.

Another embodiment of the present invention provides a method for removing ash. The method includes: receiving air into a chamber through an inlet; heating the air in the chamber by combusting fuel, thereby generating such ash; transporting the heated air out of the chamber through an outlet; and removing the ash from the chamber by blowing the ash to an edge of a floor of the chamber with a blower, wherein the blower comprises at least one conduit, and at least one nozzle coupled to the at least one conduit and adapted to blow the ash to the edge.

An opening of the at least one nozzle may be oval-shaped so that a medium from the nozzle is directed across the floor. The removing of the ash may further include transporting the ash out of the chamber with an auger.

### BRIEF DESCRIPTION OF THE DRAWINGS

FIG. 1 is a perspective view of an apparatus for sterilizing and deodorizing air according to an embodiment of the present invention.

FIG. 2 is a cross-sectional perspective view of an apparatus for sterilizing and deodorizing air according to an embodiment of the present invention.

FIG. 3 is a flow chart of a process for sterilizing and deodorizing air according to an embodiment of the present invention.

### DETAILED DESCRIPTION

The detailed description set forth below in connection with the drawings is intended as a description of embodiments of a method and apparatus for sterilizing and deodorizing air in accordance with the present invention and is not intended to represent the only forms in which the invention may be constructed or utilized. It is to be understood that the same or equivalent functions and structures may be accomplished by different embodiments that are also intended to be encompassed within the spirit and scope of the invention. As denoted elsewhere herein, like element numbers indicate like elements or features.

Some materials that must be dried to be a high grade fuel require a large quantity of high temperature air to evaporate the moisture from the material, and the air may become contaminated with odors and pathogens from the material.

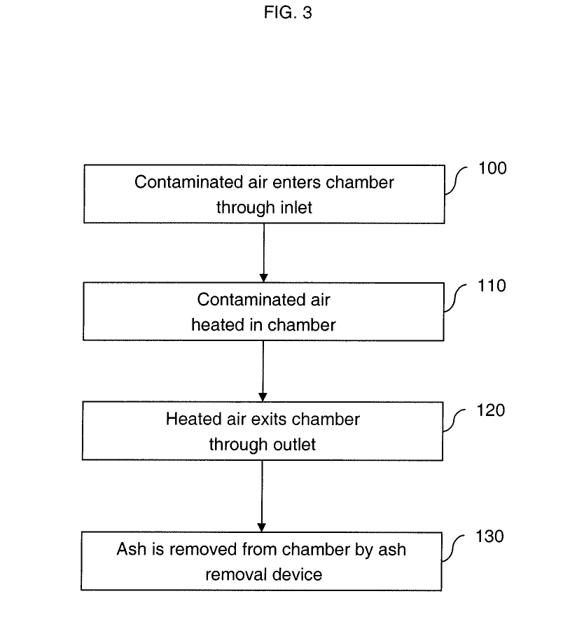

A process for sterilizing and deodorizing air according to an embodiment of the present invention is shown in FIG. 3. Here, contaminated air enters a chamber through an inlet 100. The contaminated air is heated in the chamber 110 to a suitably high temperature for a suitably long period to sterilize and deodorize the air. The heating of the air may create ash, either as a result of the combusting of fuel to heat the air or the sterilization of contaminates in the air. For example, the ash may be similar to sand.

The heated air exits the chamber through an outlet 120, where the air may be further processed or returned to the atmosphere. The ash is removed from the chamber by an ash removal device 130.

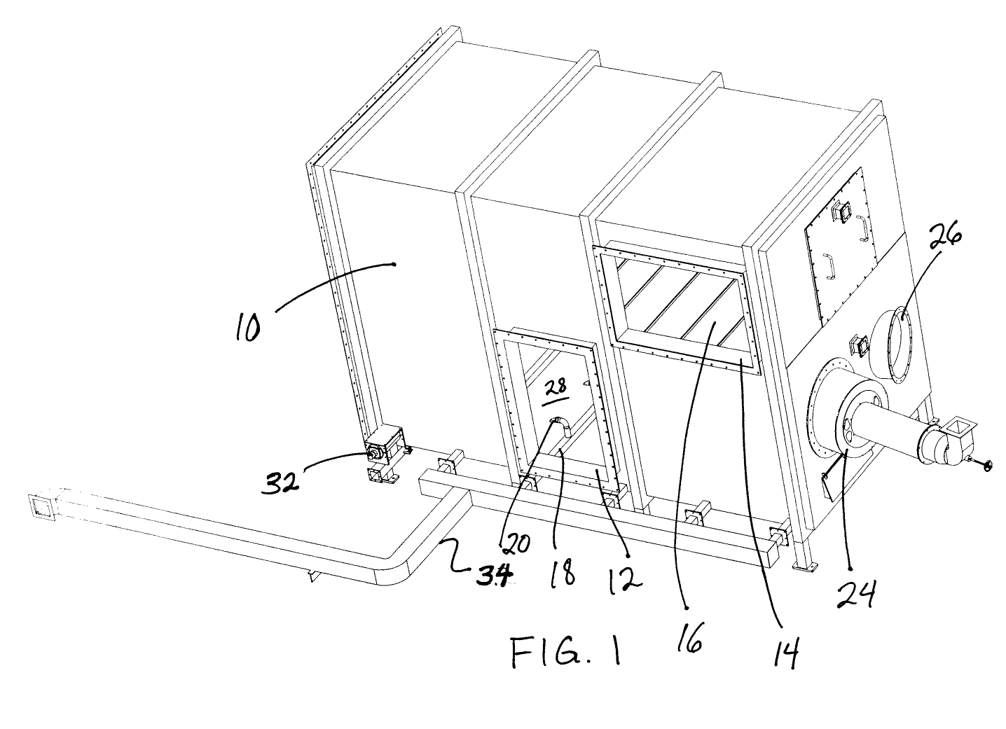

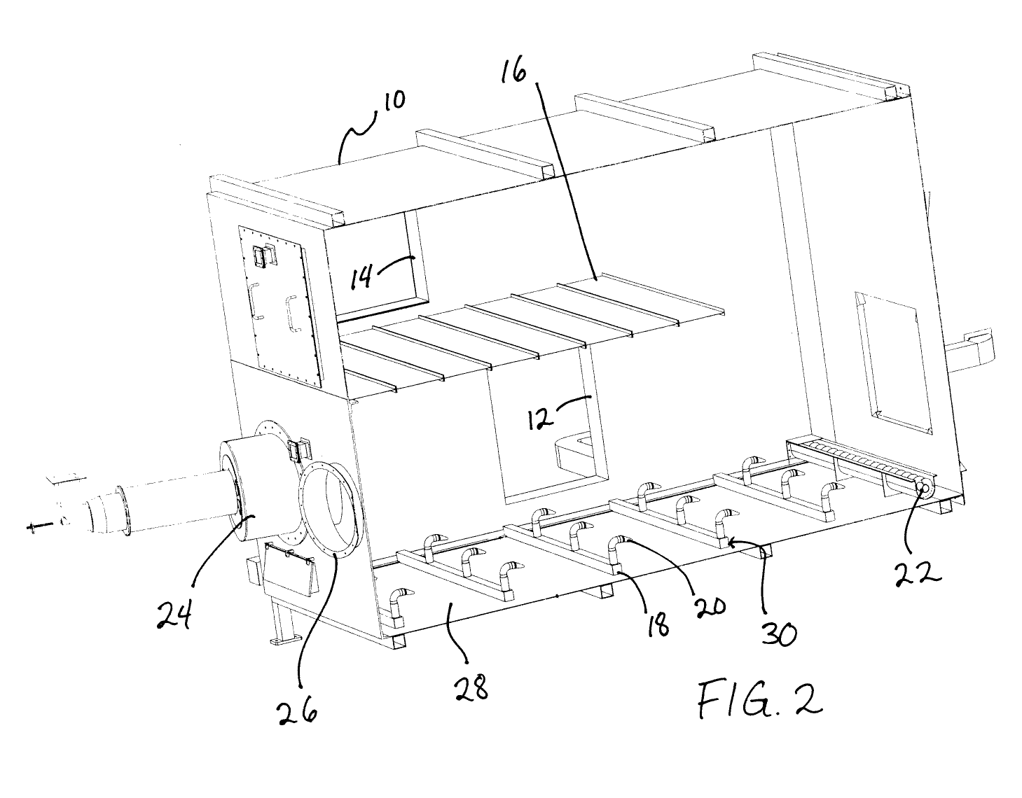

An apparatus for sterilizing and deodorizing air according to an embodiment of the present invention is shown in FIGS. 1 and 2. Here, contaminated air enters a chamber 10 through an inlet 12. The contaminated air is heated by at least one combustor 24, 26.

In an embodiment of the present invention, a combustor 24, e.g., a fuel injector or other suitable combustor, combusts powdered organic fuel to heat the air. For example, the powdered organic fuel may be generated from the material, such as sewage, that was dried with the preheated fresh air.

In an embodiment of the present invention where the material is sewage, the powdered organic fuel combusts at about 1100 degrees C. Once the powdered organic fuel is combusted, the ash left behind is basically sand, which may be utilized in many construction applications.

For example, the composition of the sand was experimentally found to be as follows:

| | |
|---|---|
| SiO₂ | 61.4% |
| Al₂O₃ | 14.1% |
| Fe₂O₃ | 5.5% |
| CaO | 4.1% |
| MgO | 1.7% |
| Na₂O | 3.4% |
| K₂O | 1.7% |
| TiO₂ | 1.0% |
| Mn₃O₄ | 0.10% |
| SO₃ | 0.30% |
| P₂O₅ | 4.10% |

In an embodiment of the present invention, an alternate combustor 26 may combust an alternate fuel, such as liquid natural gas or any other suitable fuel, if the combustor 24 is not combusting powdered organic fuel.

The heating of the contaminated air may combust particulate or contaminates in the air, which may generate additional ash.

In an embodiment of the present invention, the chamber 10 may be refractory lined. For example, the lining of the chamber 10 may have a hard surface to prevent or reduce ash deposition, such as a hard surface ceramic faced insulation.

In an embodiment of the present invention, the contaminated air is heated to a suitably high temperature to sterilize and deodorize the air, e.g., to a temperature in a range from about 800 degrees C. to about 850 degrees C.

In an embodiment of the present invention, the temperature of the air in the chamber 10 may be measured with a thermostat. For example, the thermostat may be positioned at the outlet 14 or any other suitable location.

In an embodiment of the present invention, the air travels around a baffle 16. The baffle 16 increases the length of the path traveled by the air in the chamber 10, thus increasing the length of time that the air is in the chamber 10. Here, the air in the chamber 10 travels through two passes to travel around the baffle 16.

In an embodiment of the present invention, the contaminated air is retained in the chamber 10 for a suitable length of time for the air to be sterilized and deodorized, e.g., for a time period in a range from about one to about two seconds.

In an embodiment of the present invention, the baffle 16 is horizontally positioned across part of the chamber 10. For example, the baffle 16 may be positioned so that the cross-sectional area of the chamber 10 through which the air flows or passes beneath the baffle 16 is 50% greater than the cross-sectional area through which the air flows or passes above the baffle 16. Here, the flow velocity of the air beneath the baffle 16 will be lower than the velocity of the air above the baffle 16. For example, the flow velocity of the air beneath the baffle 16 will be below the floating velocity of the ash so that the ash will drop out of suspension in the air and fall to the floor 28 of the chamber 10.

The heated air exits the chamber 10 through an outlet 14, where the air may be transported to be further processed or may be released to atmosphere.

Ash generated by the combusting of fuel and/or the combusting of particulate or contaminates in the air falls to the floor 28 of the chamber 10. The ash is then removed by an ash removal device 30.

In an embodiment of the present invention, the ash removal device 30 is a clean-in-place type device that allows for the removal of ash during the regular operation of the apparatus. Here, the ash removal device may be a blower that blows air, or another suitable gas or other medium, across the floor 28 of the chamber 10 to blow or otherwise move the ash to one edge of the floor 28.

In an embodiment of the present invention, the blower includes at least one air conduit 18 and at least one nozzle 20 coupled to the air conduit 18 and adapted to blow the ash to one edge of the floor 28. For example, the blower may include multiple (e.g., five) air conduits 18, and multiple (e.g., three) nozzles 20 may be coupled to each air conduit 18.

In an embodiment of the present invention, the opening of the at least one nozzle 18 may be oval-shaped with its longer diameter parallel to the plane of the floor so that the air blown through the nozzle 18 blows across the floor. Other shapes for the nozzle 18 suitable to directing air across the floor may also be used. Further, multiple nozzles 18 may be suitably spaced on the floor 28 so that ash is removed from the entire floor 18.

In an embodiment of the present invention, the at least one air conduit 18 is coupled to an air supply source 34. One of ordinary skill in the art will appreciate that the air utilized in the blower may be obtained from various suitable sources. For example, the air utilized in the ash removal device 30 may be air utilized in the processing of the material, or the air may be from some other suitable source.

In an embodiment of the present invention, the pressure of the air being blown from the ash removal device 30 has a suitable pressure, e.g., a suitably high pressure, to blow the ash to an edge of the floor 28. For example, the pressure of the air being blown from the blower may be about 12″ wg (inches water gauge).

In an embodiment of the present invention, an auger 22, or screw conveyor, is at the edge of the floor 28 where the ash is blown by the ash removal device 30. The ash may be blown into the auger 22, where the auger 22 removes the ash from the chamber 10 through an ash outlet 32. The auger 22 may be powered or rotated by a motor. The auger rotates or removes ash from the chamber 10 at a suitable rate so that ash does not build up in the chamber 10. Here, the ash may either be taken out of the system or further processed.

In an embodiment of the present invention, an insulation layer is on the floor 28 around the auger 22 and the ash removal device 30. For example, the insulation layer may have a thickness so that a top of the insulation layer is just below the nozzle 18 and flush with the top of the auger 22. For example, the insulation layer may be about eight inches thick, and the top of the insulation layer maybe within about ten mm from the bottom of the nozzle 18.

In an embodiment of the present invention, the auger 22 has a hollow shaft into which air may be drawn to cool the auger 22 and/or the ash.

In an embodiment of the present invention, a conduit is sealed to the inlet 12 for the delivery of contaminated air, and another conduit is sealed to the outlet 14 for the removal of heated air.

Although the present invention has been described through the use of exemplary embodiments, it will be appreciated by those of skill in the art that various modifications may be made to the described embodiments that fall within the scope and spirit of the invention as defined by the claims and their equivalents appended hereto. For example, aspects shown above with particular embodiments may be combined with or incorporated into other embodiments.

What is claimed is:

1. An apparatus for sanitizing and deodorizing air, the apparatus comprising:
a chamber comprising:
an inlet adapted to receive contaminated air,
an outlet adapted to transport heated air,
a baffle adapted to increase the length of a path traveled by air in the chamber, and
an ash removal device adapted to remove ash from the chamber; and
at least one combustor,
wherein the combustor is adapted to combust a fuel to heat the air in the chamber so that the air is sterilized and deodorized, thereby generating such ash.

2. The apparatus of claim 1, wherein the ash removal device comprises a blower adapted to blow the ash to an edge of a floor of the chamber.

3. The apparatus of claim 2, wherein the blower comprises at least one air conduit, and at least one nozzle coupled to the at least one air conduit and adapted to blow the ash to the edge.

4. The apparatus of claim 3, wherein an opening of the at least one nozzle is oval-shaped so that air from the nozzle is directed across the floor.

5. The apparatus of claim 1, wherein the ash removal device comprises an auger adapted to transport the ash out of the chamber.

6. The apparatus of claim 1, wherein the at least one combustor comprises a combustor adapted to combust powdered organic fuel.

7. The apparatus of claim 1, wherein the at least one combustor comprises an alternate combustor adapted to combust an alternate fuel.

8. A method for sanitizing and deodorizing air, the method comprising:
receiving contaminated air into a chamber through an inlet;
heating the contaminated air in the chamber by combusting fuel with at least one combustor to sterilize and deodorize the air, thereby generating ash, wherein the chamber comprises a baffle adapted to increase the length of a path traveled by the air in the chamber;
transporting the heated air out of the chamber through an outlet, and
removing the ash from the chamber with an ash removal device.

9. The method of claim 8, wherein the removing the ash comprises blowing the ash to an edge of a floor of the chamber with a blower.

10. The method of claim 9, wherein the blower comprises at least one air conduit, and at least one nozzle coupled to the at least one air conduit and adapted to blow the ash to the edge.

11. The method of claim 10, wherein an opening of the at least one nozzle is oval-shaped so that air from the nozzle is directed across the floor.

12. The method of claim 8, wherein the removing the ash further comprises transporting the ash out of the chamber with an auger.

13. The method of claim 8, wherein the at least one combustor comprises a combustor adapted to combust powdered organic fuel.

14. The method of claim 8, wherein the at least one combustor comprises an alternate combustor adapted to combust an alternate fuel.

15. An apparatus for removing ash, the apparatus comprising:
a chamber comprising:
an inlet adapted to receive air,
a combustor adapted to heat the air, thereby generating such ash,
an outlet adapted to transport heated air, and
a blower adapted to blow the ash to an edge of a floor of the chamber wherein the blower comprises at least one conduit, and at least one nozzle coupled to the at least one conduit and adapted to blow the ash to the edge.

16. The apparatus of claim 15, wherein an opening of the at least one nozzle is oval-shaped so that a medium from the nozzle is directed across the floor.

17. The apparatus of claim 15, the chamber further comprising an auger adapted to transport the ash out of the chamber.

18. A method for removing ash, the method comprising:
receiving air into a chamber through an inlet;
heating the air in the chamber by combusting fuel, thereby generating such ash;
transporting the heated air out of the chamber through an outlet; and
removing the ash from the chamber by blowing the ash to an edge of a floor of the chamber with a blower, wherein the blower comprises at least one conduit, and at least one nozzle coupled to the at least one conduit and adapted to blow the ash to the edge.

19. The method of claim 18, wherein an opening of the at least one nozzle is oval-shaped so that a medium from the nozzle is directed across the floor.

20. The method of claim 18, wherein the removing the ash further comprises transporting the ash out of the chamber with an auger.
